(19) United States
(12) Patent Application Publication
(10) Pub. No.: US 2023/0070616 A1
(43) Pub. Date: Mar. 9, 2023

(54) System and Method for More Accurate Estimation of Vaccine Efficacy by Taking Into Account the Rate of Herd Immunity

(71) Applicants: George R. Oliver, Southlake, TX (US); Steve Miff, Dallas, TX (US); Thomas Roderick, Frisco, TX (US)

(72) Inventors: George R. Oliver, Southlake, TX (US); Steve Miff, Dallas, TX (US); Thomas Roderick, Frisco, TX (US)

(21) Appl. No.: 17/939,921

(22) Filed: Sep. 7, 2022

Related U.S. Application Data
(60) Provisional application No. 63/242,007, filed on Sep. 8, 2021.

(51) Int. Cl. G16H 50/80

(52) U.S. Cl. CPC G16H 50/80

(57) ABSTRACT

A system comprises a data ingestion logic configured to receive pandemic data associated with susceptible, exposed, infected, recovered, and vaccinated people in a community; a data analysis logic configured to weigh, normalize, calculate, or use artificial intelligence to analyze the received pandemic data; and a dashboard configured to visually present values for a plurality of indicators; wherein the data analysis logic includes a herd immunity module determining values associated with an accurate estimation of herd immunity.

### RELATED APPLICATION

This patent application claims the benefit of U.S. Provisional Patent Application No. 63/242,007 filed on Sep. 8, 2021, the entirety of which is incorporated herein by reference.

### FIELD

This disclosure relates to a system and method for a more accurate estimation of vaccine efficacy by taking into account of community herd immunity for a highly contagious disease.

### BACKGROUND

Herd immunity occurs when a large portion of a community becomes immune to a disease. The spread of disease from person to person becomes unlikely when herd immunity is achieved. As a result, the whole community becomes protected—not just those who are immune.

The percentage of a community that needs to be immune in order to achieve herd immunity varies from virus to virus, disease to disease. Generally, the more contagious a disease is, the greater the proportion of the population that needs to be immune to the disease to stop its spread. There are several ways to achieve herd immunity: vaccines, natural infection, or hybrid immunity obtained by both natural infection and vaccination.

### BRIEF DESCRIPTION OF THE DRAWINGS

A better understanding of the claimed subject matter can be obtained when the following detailed description of the disclosed embodiments is considered in conjunction with the following figures.

FIGS. 1A and 1B are Venn diagrams that graphically illustrate the complexity of Covid-19 community immunity components;

FIGS. 2-4 are graphic plots of data related to Covid-19 cases;

FIG. 5 is an illustration of Covid-19 data sources;

FIG. 6 is a simplified block diagram of an example system and method for determining a more accurate estimation of vaccine efficacy;

FIG. 7 is a simplified block diagram of an example system and method for determining a community vulnerability index that may be used to inform the calculation and analysis done in the herd immunity estimation system and method;

FIG. 8 shows the equations used to determine herd immunity;

FIG. 9 is the flowchart for current Covid-19 vaccine effectiveness;

FIG. 10 is a chart illustrating how the equation estimates time efficacy of vaccine protection in population;

FIG. 11 is a simplified block diagram of an example system and method for determining a more accurate estimation of vaccine efficacy;

FIG. 12 is a simplified block diagram of the hardware components of an embodiment of the personal pandemic proximity index system and method according to the teachings of the present disclosure; and

FIG. 13 is a simplified block diagram of the exemplary operating environment of the personal pandemic proximity index system and method.

### DETAILED DESCRIPTION

It has been recognized that a system and method for more accurate estimation of vaccine efficacy that takes into account of herd immunity or community infection is needed for an effective disease prevention strategy. Management of pandemic spread (e.g., Covid-19) has tested the healthcare, political, and social fabric of communities around the world. For all, the term “flatten the curve” has come to represent how specific measures, such as vaccination and masking, can slow the spread of the virus, which in turn can help to mitigate the tidal surge that would overwhelm the capacity of the healthcare system. For any region, there is a critical window of opportunity to leverage advanced analytics, geospatial modeling (hot-spotting), and integrated patient management tools to better equip civic leaders and care delivery teams with real-time information to mitigate the surge and save more lives. With the introduction of vaccines, emergence of new virulent strains, and occurrence of vaccine breakthrough cases, the attempt to depict an accurate Covid-19 SEIRV (susceptible, exposed infectious, recovered, vaccinated) picture has become even much more challenging. Current estimates of vaccine efficacy are subject to an artificial decrease due to the increasing fraction of unvaccinated patients infected at a historic pace. Wide-ranging local variations in vaccination rates, infection rates, and population behaviors will, together, affect population immunity. The United States needs a better understanding of local pandemic conditions to create more focused and locally-tailored strategies and responses. A more accurate estimation of vaccine efficacy that takes into account of herd immunity or community infection rates enables civic leaders and communities to design and deploy culturally and politically-sensitive and effective interventions to better motivate and encourage vaccination and protective behavior compliance for certain communities.

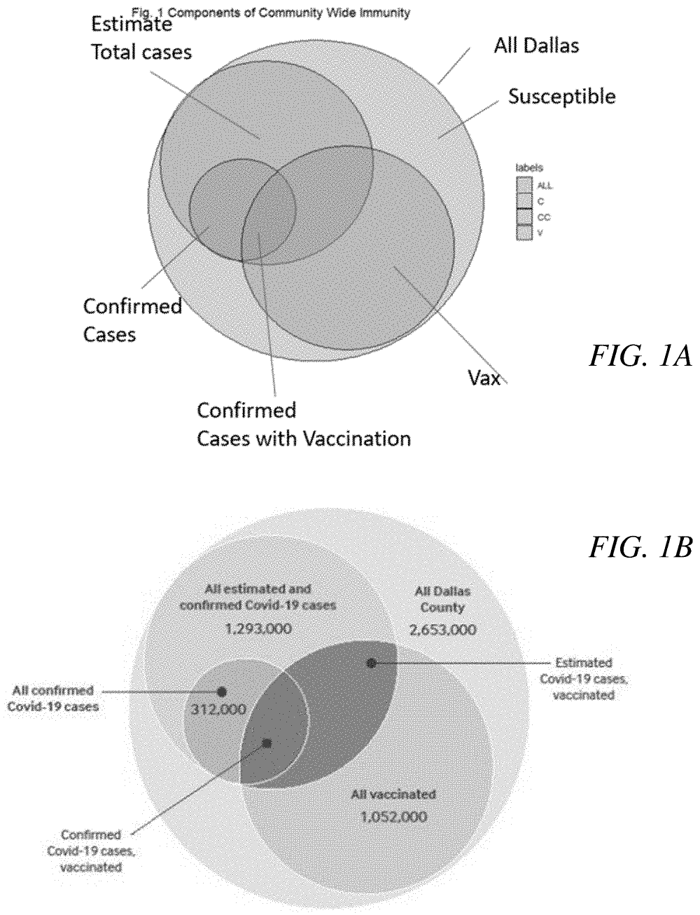

FIG. 1A-1B are Venn diagrams depicting the complexity of Covid-19 immunity. There is a gap between the estimated infected cases and the actual confirmed cases; and the estimated Covid-19 infected cases with vaccination and the confirmed cases with vaccination. Not all people who are vaccinated achieve effective immunity. As shown in FIG. 1B, there are 1,293,000 estimated and confirmed Covid-19 cases, but the confirmed Covid-19 cases are only 312,000. The data difference shows the complexity of monitoring Covid-19 spread and estimating the efficacy of vaccines.

Dallas County has implemented a system to track the efficacy of vaccines on healthcare utilization of new diagnosed cases, hospitalization and ICU utilization from Jan. 1, 2021 to Aug. 31, 2021. Measured vaccine efficacy 42 days after first dose injection for all vaccines is 95, for mRNA vaccines is 99, and protective immunity from confirmed prior testing is 98 compared to the estimated population of Dallas subtracting known vaccinated patients and prior confirmed infection. For the susceptible-exposed-infected-removed-vaccinated (SEIRV) framework, S is a value populated from Dallas County ACS2019 accessed through tidy census; I is confirmed or probable cases reported as Dallas County resident to DCHHS infection documented by Ag testing or PCR; V is vaccinated population as documented in Texas State ImmTrac2 database for Dallas County residents; V→I is data for vaccine breakthrough cases reported to DCHHS≥42 days first dose of vaccination to account for higher rates of infections in Johnson and Johnson vaccination group using CDC definition and to allow analysis of patients with partial vaccinations; R is data for infected and recovered population; R→I is data for the reinfected.

R→I (filtering I<90 days after initial infection)/R: S→1/S (removing confirmed I and V)

V→I/V(≥42 days from 1st dose of mRNA vaccine): S→1/S (removing confirmed I and V)

Herd immunity is reached when a sufficient proportion of the community in a defined geographical area develops a threshold level of immunity to new infection that prevents exponential spread of infections in that community (i.e., the “effective reproduction number” [Ro]—the number of community members infected by each case of Covid-19 is consistently≤1). When herd immunity is achieved, new infections appear in that community at a very low rate and are primarily due to the introduction of new infections from outside that community. Because herd immunity depends on biological and behavioral factors that are ever-changing and context-specific, the precise threshold for herd immunity for a given population in a specific geography is difficult to determine. Four factors determine community-wide immunity:

Infection-related immunity: Covid-19 immunity can be acquired through prior exposure to the virus, both known and unknown. This type of immunity can be determined by using records of positive Covid-19 tests and estimates of the ratio of undiagnosed to diagnosed infections, which is estimated to be between ˜1.8 and 12.2 for different regions of the U.S. as of the summer of 2020. The extent of immunity conferred by prior Covid-19 infection also may vary according to the dose of the inoculating infection (reflected by symptomatic or asymptomatic infections), host response (e.g., age, immune status), and infection variant (e.g., Alpha, Delta, Lambda). The duration of immunity resulting from prior infection is unknown, and there are emerging concerns that immunity derived from prior exposure to initial Covid-19 genotypes may provide variable or limited protection from recent and future variants, which have the potential to displace circulating viruses and become predominant.

Vaccine-related immunity: Vaccination rates for the thousands of counties in the U.S. are highly variable, both between states (e.g., 33% of individuals being fully vaccinated in Mississippi, compared with 66% in Vermont) and within states (e.g., 21% to 65% of individuals being fully vaccinated across counties in North Carolina), with further variations by age, race, socioeconomic status, and political affiliation. The effectiveness of vaccine-induced immunity depends on the type of vaccine used (with all three vaccines available for use in the U.S. having very high rates of protection against serious infection and imperfect protection against transmission), the prevailing Covid-19 variant in that location, and the time elapsed since the last dose of vaccine. As of the summer of 2021, vaccines used in the U.S. were highly effective for protecting against serious disease from the current Covid-19 variants, and vaccine immunity is expected to be sustained for several months.

Opportunity for infection: For fully vaccinated Americans, the Centers for Disease Control and Prevention has issued changing guidelines related to mask-wearing and physical distancing as the emerging threat from Covid-19 variants has become better understood. On the other hand, many states, including school districts, have abandoned most, if not all, public health measures for Covid-19, except for large indoor gatherings. In areas with fewer preventive measures, there is increasing opportunity for ongoing community infection. This is true at a time when the new variants (e.g., Delta, Omicron) predominate among both vaccinated and unvaccinated people.

Infectiousness of the virus: Notwithstanding the difference in population behavior (e.g., whether a community is in “lockdown” or people are moving about without restriction), the infectiousness of the virus will also determine a threshold for the proportion of the population that needs to be immune in order to reach herd immunity. For the initial Covid-19 genotypes, that threshold was thought to be around 70%; for the Delta variant, the threshold is thought to be near 90%. This value could be higher still if the Ro of the Delta or other variants prove to be higher still.

The interplay between these four factors can help us understand the extent of projected Covid-19 community protection in the U.S. and direct more locally targeted efforts to protect communities at greatest risk. Each locally defined population (e.g., county, ZIP code, or census tract) will include an overlapping mix of unvaccinated, partially vaccinated, fully vaccinated, and known/unknown Covid-19-exposed residents. Some counties are already using this more nuanced thinking to determine local community immunity levels and target their actions.

The Parkland Center for Clinical Innovation in Dallas County, Tex., has used known vaccination rates, records of prior infections along with estimates of unknown infections, and, importantly, an approximation of the overlap of these factors to estimate population immunity for the county's 94 ZIP codes. Despite achieving full vaccination for under 40% of the population, Dallas County estimated that by Jul. 4, 2021, Covid-19 immunity existed for greater than 80% of the population. This value represents the threshold of population immunity that was estimated to be necessary in order to stop community spread of Covid-19 variants circulating at that time. However, while the overall county estimates of immunity were high, populations in 45 ZIP codes were under the 80% threshold (with eight communities having <50% immunity). This wide variation in immunity rates points to significant pockets of populations within Dallas County that were vulnerable to increasing rates of new infections. The hyper-localized herd immunity calculations as well as data on the individual components of herd immunity in Dallas County are being used by local health providers, county health departments, and local municipalities to (1) identify locations with proportionately lower immunity and vaccination rates and (2) prioritize them for coordinated outreach and vaccination site deployment efforts. The data that are used to track progress of these efforts and to estimate changing protection against variants are communicated across health entities and to the public.

Community-wide immunity is determined by using individual identifiable data across vaccinations and measured and reported infections. Community-wide immunity is calculated from the sum of the vaccinated individuals and Covid-infected individuals, known through testing and estimated by seroprevalence, correcting for overlap. The estimate of total cases is derived from the total number of confirmed cases multiplied by the local estimate of the adjusted incidence rate ratio (AIRR). There is an increasing number of individuals who had been previously diagnosed with Covid-19 (confirmed) and subsequently received a vaccine. To account for the overlap in these populations, the data sources for total confirmed cases and vaccinated cases are statistically matched on the basis of demographic information and geocoded to be a resident of Dallas County using the Azure BING APL. The following equation is used in the community-wide immunity calculations:

HI=1−S/T,

where S(θ)=T−H=T−(θ[I+R]+[I−λ]V).

S=susceptible; T=total; V=vaccinated people, accounting for the effective model; I=measured infected (14-day rolling case count); R=measured recovered (cases no longer in 14-day window); θ=measurement gap, the estimate count of total cases per each observed case; λ=overlap percent, a multiplicative factor representing the number of people who are vaccinated who had the virus previously; and HI=herd immunity, the proportion of the population that has some immunity to the virus, expressed as a percentage.

The immunity generated from Covid-19 by vaccination differs from a naturally acquired infection. Protective antibodies generated in response to a vaccine will target virus carrying “single letter” changes in the receptor binding domain of the spike protein compared to antibodies acquired from an infection which targets both the receptor binding domain and other portions of the spike protein.

Overlap percent λ is defined from serology and blood sample studies in a percent of population proportion. Overlap is the population both in vaccinated and infected groups. [1-λ]V represents the number of cases that only acquired immunity via Covid-19 vaccines. For Dallas County, overlap is defined at 78.4%. Overlap is constant across individual states. Missing states are filled with U.S. average. At present, all states are reporting.

I+R represents the number of reported cases immunized via natural infection. θ[I+R] represents the number of estimated cases immunized via natural infection. Measurement gap θ allows us to compare the incident rate between two different groups: the reported infected cases and the estimate infected cases including unreported cases. Measurement gap θ is also defined from serology and blood sample studies. Measurement gap θ=the estimate of total infected cases/the number of measured infected cases. A small adjustment is applied to Dallas County to account for more available test reporting than rural area in Texas. The Dallas County rate is pegged at 2.39 cases per reported case. Measurement gap θ is constant across states. Missing states, including Montana and New Hampshire, are filled with U.S. average. The measurement gap θ varies in different time windows. In the equation above, the time window is 14-day. When applied to a defined time window, the measurement gap θ will be referred to as Adjusted Incidence Rate Ratio (AIRR).

The term θ[I+R]+[I−λ]V represents the number of people who are immunized against Covid-19. T−(θ[I+R]+[I−λ]V) represents the number of people who have never exposed to Covid-19 via a vaccine or an infection, making them susceptible to Covid-19. By measuring people with immunity against Covid-19, the equation (HI=1−S/T) measure the level of herd immunity in a community.

A first caveat to this analysis is that it is assumed that both prior infection and vaccination provide close to full immunity. This assumption does not hold for communities predominantly infected by the Delta variant or other variants against which infection-induced and vaccine-induced immunity provide diminished protection. In the face of evidence of diminished protection of vaccines over time and against new variants, booster vaccine doses are now being recommended, and adapted vaccines may be required in the future to achieve adequate immune protection.

A second caveat is the rapid dominance of the Delta variant in Dallas County over time. In June 2021, when the estimated threshold of 80% was reached, the county had low rates of new infections (about 5 new cases per 100,000 population per day). At the time of the calculation, the proportion of Delta variant was estimated to represent 30% to 45% of all Covid-19 cases. As of mid-August 2021, the proportion new Covid-19 infections due to the Delta variant in the U.S. had risen to over 98%, moving the herd immunity threshold to close to or above 90%. As the fraction of population infected by the Delta or other variants increases, and as the degree of immune protection of previously infected and vaccinated residents decreases, the threshold for herd immunity increases. This increase in herd immunity threshold is occurring while the existing immunity of local populations that “counts” towards herd immunity decreases. Moreover, the vulnerability of the partially immunized and non-immunized sectors and the extent of public health precautions followed by the local population could vary, further complicating the calculation of herd immunity.

Herd immunity is dynamic. The threshold for herd immunity changes, depending on the transmissibility of each new Covid-19 strain and the effectiveness of previous immunity, both from previous infection and from vaccination to these strains, as well as behaviors within local communities. The Dallas example took into account every Covid-19 variant that was locally prevalent at the time. Through June 2021, immune protection of the community, reflected by low rates of new infections, stayed strong. The dominance of the Delta variant changes the estimates of population immunity in two ways. First, there is greater transmissibility, with an increase in Ro from 3-5 for the Alpha variant to 5-8 for the Delta variant. This shift in Ro increases the threshold estimates required for community-wide immunity to as high as 97%. Second, given the concerns that the newer variants can evade natural and vaccine-induced immunity to varying degrees, less immunity can be assumed for those who have been fully vaccinated (70% to 90% protection) or previously exposed to Covid-19 infection (60% to 80% protection), particularly those who are presumed to be in the asymptomatic infection group.

In the face of very high thresholds for herd immunity, diminishing protection from vaccines and infections, and ongoing resistance to vaccination, true uniform herd immunity to Covid-19 would be difficult to achieve in the near future. The key to future control of a chronic Covid-19 pandemic may rest with a combination of a more nuanced and highly localized understanding of population immunity and behaviors. Community-wide immunity in a particular location cannot be inferred only from the vaccine status of larger populations. Assumptions also cannot be made that exposed populations will automatically be protected through prior Covid-19 infections. The impact of relaxing Covid-19 public health protections may have on community-wide immunity cannot be discounted. In this highly dynamic environment, local coalitions of county, healthcare, business, and community stakeholders can use real-time, highly localized and contextualized data to target local actions and communications that promote vaccination and public health protections. It is more important than ever to build highly local situational awareness for local coalitions of decision-makers, who can create interventions and communications that respond adaptively to local pandemic conditions in real time.

Given the complexity and local variation of population immunity to Covid-19, we propose the following actions:

Remain focused on achieving very high levels of Covid-19 vaccinations in every community as the primary tactic for increasing population immunity.

Take into account both vaccination rates and known and estimated prior infection rates when assessing population risk from Covid-19 in local communities.

Retain public health protective measures, especially in enclosed spaces, for the foreseeable future.

Empower local partnerships, committed to local action and informed by learning from timely local data, to deliver these interventions to the communities they know best.

A real-time dashboard may display a heat map of the geographical region in question. The map shows geospatially each of the herd immunity components (SEIRV) distributed in the region and how the percentages are evolving. The map may have overlay layers that show locations of hospitals, testing centers, and vaccinations to assess whether community needs are being addressed.

The present system and method may incorporate machine learning and artificial intelligence in data analysis to derive the data. The herd immunity data is presented graphically with sufficient granularity to aid in comprehension and incorporated in clinical workflows to make them actionable. The herd immunity dashboard generates highly specific, block group-level indicators from a variety of publicly available data sources and present the data analysis via a highly interactive and user-friendly geospatial graphical user interface that are adaptable for a variety of computing platforms. The herd immunity dashboard provides actionable insights that enable community and civic leaders to more optimally deploy and manage physical distancing and quarantine tools.

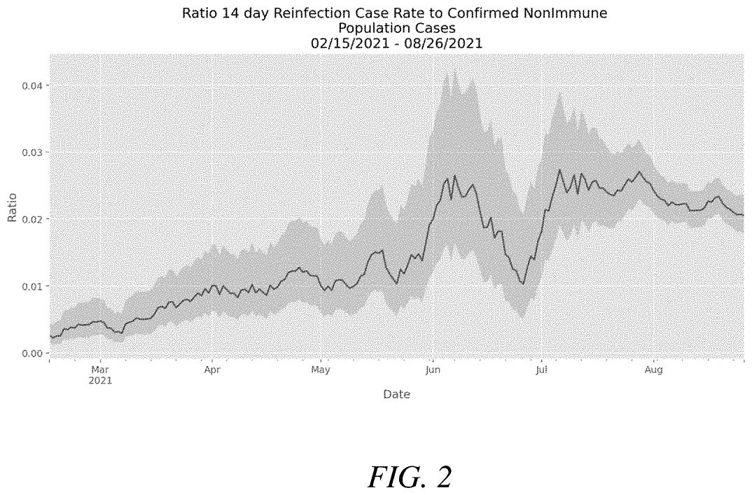

FIG. 2-4 are graphic plots of data related to Covid-19 cases. FIG. 2 illustrates the graphic plot of the ratio (14-day reinfection case count/14-day confirmed nonimmune population cases) during Feb. 15, 2021 to Aug. 26, 2021. FIG. 3 illustrates the ratio (14-day post-two shot vaccination breakthrough case/14-day confirmed nonimmune population cases) during Feb. 15, 2021 to Aug. 26, 2021. FIG. 4 illustrates the ratio (14-day post vaccination breakthrough case rate to confirmed nonimmune population cases) during Feb. 15, 2021 to Aug. 26, 2021. The three graphic plots show that rates of Covid-19 cases were lower among fully vaccinated persons with two shots, compared with people without fully vaccination and people who acquired immunity via infection. The three plots also show that efficacy of infection-acquired immunity and vaccine-acquired immunity changes over time, but the rate is more stable in post-two shot vaccination population.

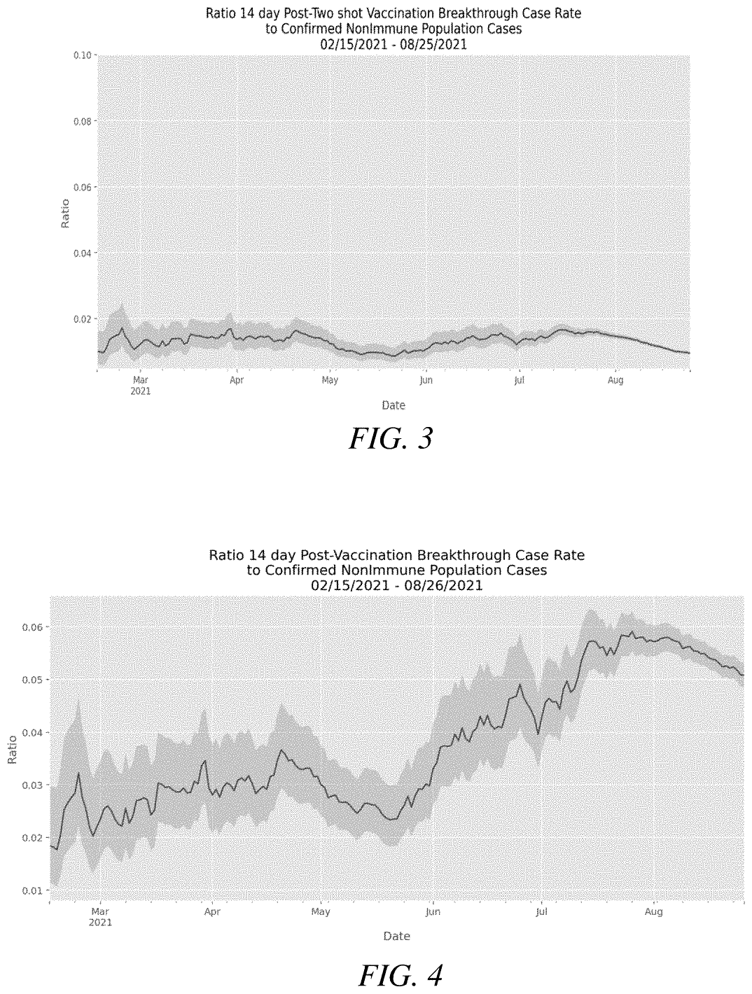

FIG. 5 illustrates Covid-19 data sources. The data sources used herein include both publicly available and proprietary/licensed data such as the census and pandemic tracking governmental sites, Facebook data, Google data, Apple data, SafeGraph data, Hospital data, jail health data, employee benefits and plan data, homeless shelter and food bank data, and nursing home data.

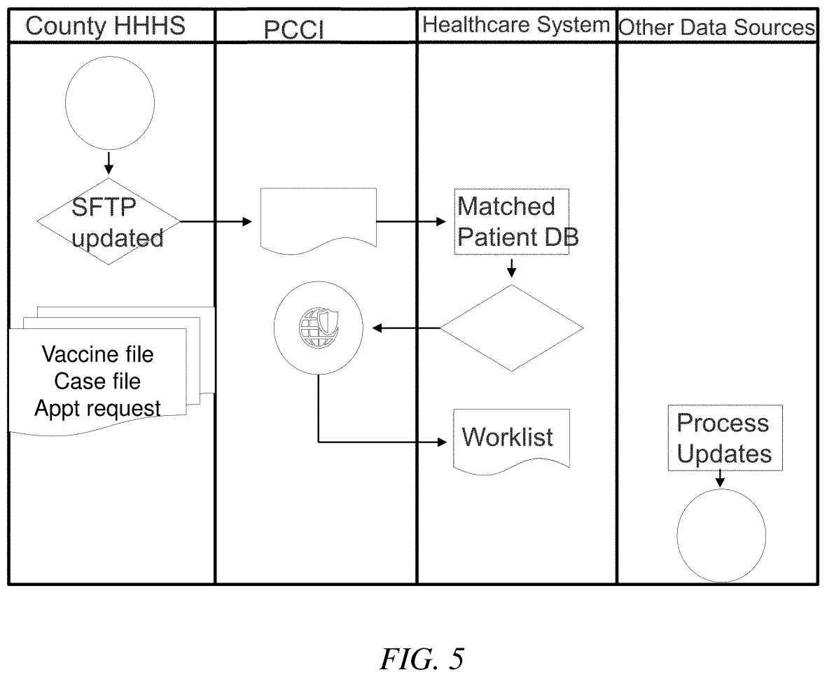

FIG. 6 is a simplified block diagram of an example system and method for determining a more accurate estimation of vaccine efficacy. The system and method analyze and visually present real-time vaccination data 10, real-time testing data 12, and real-time clinical/claims data 14 that enable users to truly understand factors that impact the herd immunity level, the vaccine efficacy, and the disease trend. The herd immunity estimation results can be displayed in heatmap, ranked list, scatter plot, or other designated manners.

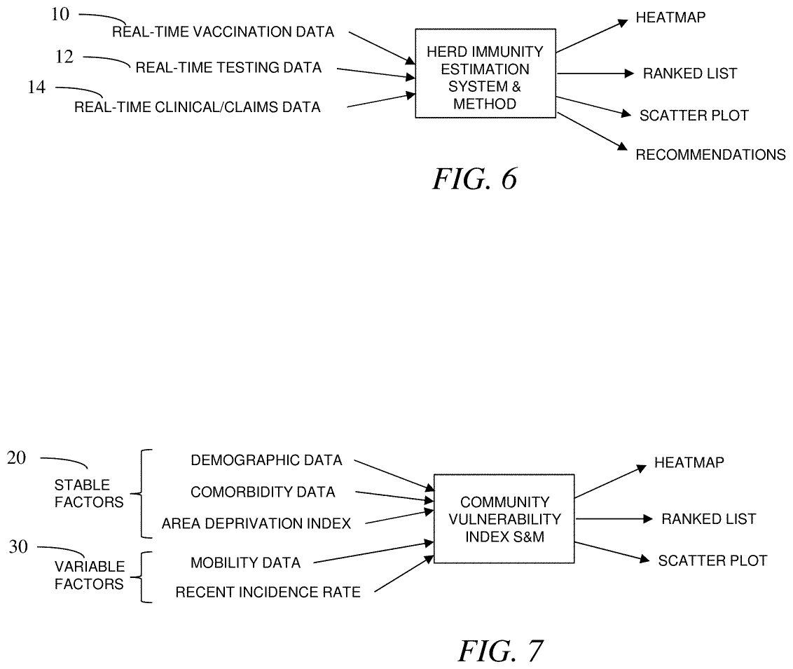

FIG. 7 is a simplified block diagram of an example system and method for determining a community vulnerability index that may be used to inform the calculation and analysis done in the herd immunity estimation system and method. The system and method use overall community factors: stable factors 20 and variable factors 30. Stable factors are made up of three sub-factors: demographic data, comorbidity data, area deprivation index. Variable factors include mobility data and recent incidence rate. By systematically analyzing both stable factors and variable factors, the system and method provide an actionable insight that enables community-based organizations and local civic leaders to interactively view Covid-19 data in a visualization manner, such as heatmap, ranked list, scatter plot. These data enable community and civic leaders to assess community herd immunity level based on various factors including demographic, area, mobility, and the likes, evaluate the effectiveness of disease prevention measures, estimate vaccine efficacy, redirect funding, track the herd immunity data, and monitor and forecast trends. The interactive dashboard provided by the system and method can also be incorporated into other use cases such as predictive models for health services utilization, neighborhood health quality index, and the impact of relaxing public health protections.

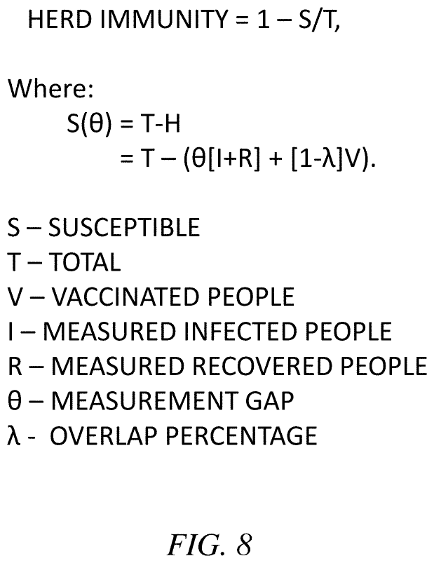

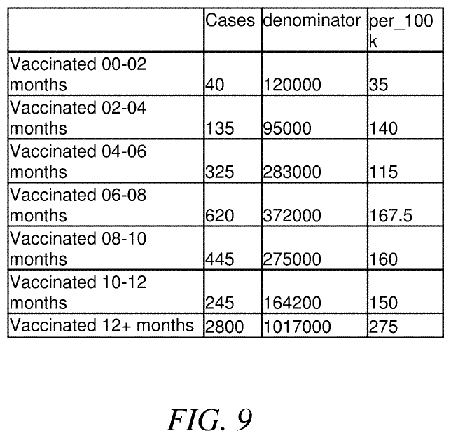

FIG. 8 is an equation used in community-wide immunity calculations. Users can designate different time windows. The equation has been discussed in prior paragraphs. FIG. 9 is a table that illustrates how efficacy of vaccine protection with the observed population varies over time. Time appears to be one of the important drivers of the post-vaccination reduction in effectiveness, as demonstrated from the chart that the infection rate after 12 months is almost two times of the infection rate in the second to fourth month. The window of immunity enabled by vaccines could have an impact on efforts to establish effective herd immunity and influence. By setting different time windows, the Adjusted Incidence Rate Ratio in the equation will change accordingly.

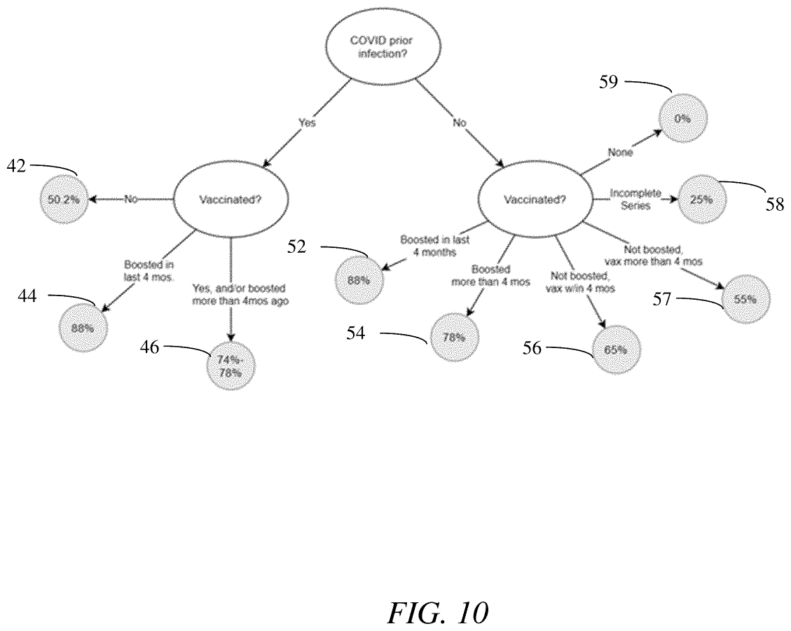

FIG. 10 is a flowchart that illustrates how the infection rate is impacted by waning factors. No vaccination and no prior Covid-19 infection 59 has no protection against Covid associated hospitalization. The efficacy of vaccine protection decreases over time. Waning factors include whether a booster is delivered in last four months, whether the population was previously infected, and whether the population is fully vaccinated. Waning factors are based on Omicron effectiveness reported by the following listed sources. According to a CDC Morbidity and Mortality Weekly Report, during the period of Omicron predominance, vaccine effectiveness against Covid-19-associated hospitalizations waned with time since vaccination: vaccination effectiveness among those not boosted and vaccinated within four months 56 declined from 65% to 55% among those not boosted and vaccinated more than four months ago 57. Vaccine effectiveness against Covid-19-associated hospitalization among people boosted in last four months without prior Covid-19 infection 52 declined from 88% to 78% among those boosted more than four months 54. The vaccine effectiveness against Covid-19-associated hospitalization among people receiving incomplete series (single dose vaccine) 58 is 25% based on an assumption from a UK Health System source. The effectiveness of Covid-19 prior infection and no vaccination against symptomatic infection 42 was 50.2%, and the effectiveness of prior infection and three doses of vaccine more than four months 46, also known as the overlap effectiveness, was 74% based on a health journal. The effectiveness of Covid-19 prior infection and boosted in last four months 44 was 88%.

The STIM implementation (Civitas) currently has Dallas County at 59.7 community protection. The ZIP Code based model has Dallas County at 60.3% community protection. The difference is driven by the difference in AIRR. This model considers waning factors and is about 6% lower than Dallas County report with the model not accounting for waning factors.

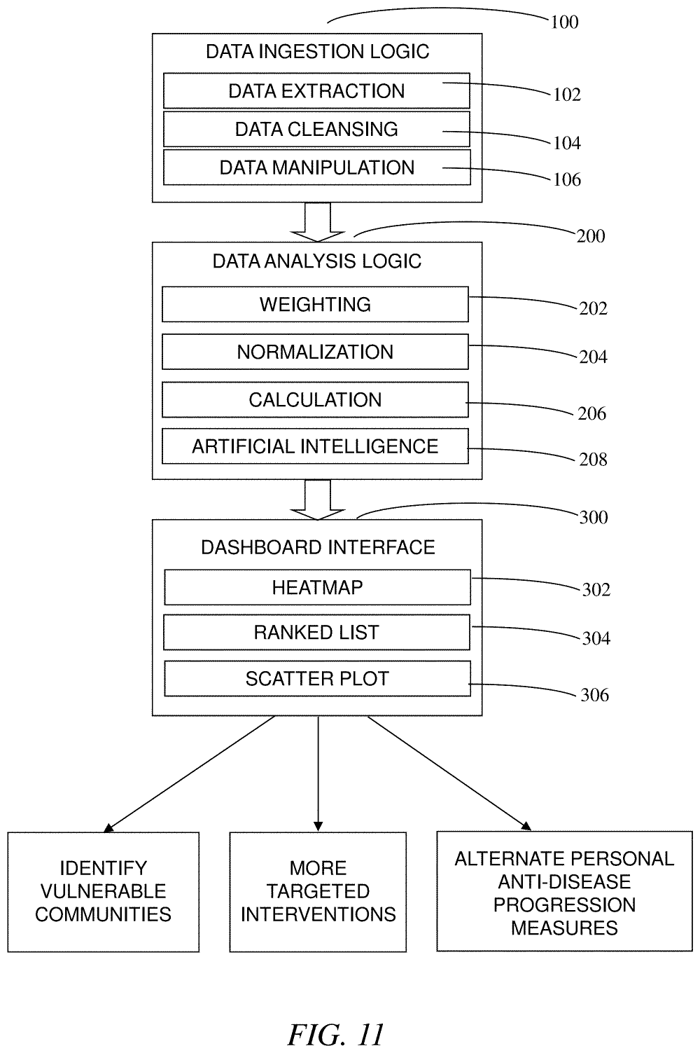

FIG. 11 is a simplified block diagram of an example system and method for determining a more accurate estimation of vaccine efficacy. Through an automated pipeline using, e.g., Apache NiFi, raw data is received by the Azure Blob Storage, using File Transfer Protocol (FTP), Simple Object Database Access (SODA)/Application Program Interface (API), client URL (cURL), and other methods.

The data ingestion logic 100 includes a data extraction module/process 102, data cleaning module/process 104, and data manipulation module/process 106. Data is automatically pulled from the sources on a regular basis to ensure that the current data is the most up to date. The data extraction module/process 102 may extract data using various technologies and protocols. The data cleaning module/process 104 “cleans” or pre-processes the data, putting structured data in a standardized format and preparing unstructured text for natural language processing (NLP) or Artificial Intelligence (AI) processing. The data manipulation module/process 106 may analyze the representation of a particular data feed against a meta-data dictionary and determine if a particular data feed should be re-configured or replaced by alternative data feeds.

The data analysis logic 200 has a weighing module/process 202, normalization module/process 204, calculation module/process 206, and an artificial intelligence module/process 208. The data analysis logic 200 uses an equation-based model, the basic units of information are weighed and normalized. The basic unites are given particular weights or importance to each variable and are calculated using the equation-based module. AI examines vast amounts of data and find the trends and patterns, develop forecasts and analyze potential scenarios, streamline data analysis by funneling all data into one solution. AI may use Tableau, Qlik Sense, Sisense, Power BI, SAS BI, or Google Data Studio. A dashboard interface 300 displays the analysis in various visualizations, including heatmap 302, ranked list 304, and scatter plot 306.

In one embodiment, the Blob Storage cleans the data for quality and accuracy according to predefined scripts. Cleaned data are then moved within the Azure environment to the PostgreSQL database management system and stored in a tabular format. The herd immunity dashboard uses the Power BI dashboard tool to pull data from the PostgreSQL database management system. Power BI is a Microsoft product and easily integrates with the Azure platform. The Power BI dashboard can be embedded into a herd immunity web portal, where users can use it to gain actionable insights into the Dallas community (or other geographical/geopolitical regions). Four dashboards were set up based on use cases identified by external and internal stakeholders, including education, community needs, health, and economy and public safety. Additionally, a master dashboard was created to provide users the opportunity to look at how indicators interact across categories. The dashboard also uses a mapping application, MapBox, that integrates well into the Power BI platform.

The data analysis logic use a predictive model mentioned above: HI=1−S/T, where S(θ)=T−H=T−(θ[I+R]+[I−λ]V). The model analyzes the data and predict the level of herd immunity. It may be used to assess the vulnerable communities, determine more targeted interventions, and design alternate personal anti-disease progression measures. The data analysis logic may use one or more models to analyze the data and calculate variables associated with herd immunity in order to more accurately predict and determine the best course of action to take with respect to a community or a disease.

Artificial Intelligence (AI) or Natural Language Processing (NLP) is used to analyze the ingested data. NLP is used, for example, to process raw data pulled from Dallas County Health and Human Services (DCHHS) and Covid-19 disease registry. The AI module or process may be used to analyze the ingested data. The AI module or process utilizes adaptive self-learning capabilities using machine learning technologies. The capacity for self-reconfiguration enables the system and method to be sufficiently flexible and adaptable to detect and incorporate trends or differences in the underlying patient data or population that may affect the predictive accuracy of a given model. The AI module or process may periodically retrain a selected model for improved accurate outcome to allow for selection of the most accurate statistical methodology, variable count, variable selection, interaction terms, weights, and intercept for a community or a local health system. The AI module or process may also adjust the predictive weights of the variables without human supervision, adjust the threshold values of specific variables without human supervision, or evaluate new variables present in the data feed but not presently used in the predictive model. The AI module or process may compare the actual observed outcome of the event to the predicted outcome, then separately analyze the variables within the model that contributed to the incorrect outcome. It may then re-weigh the variables that contributed to this incorrect outcome, so that in the next reiteration those variables are less likely to contribute to a false prediction.

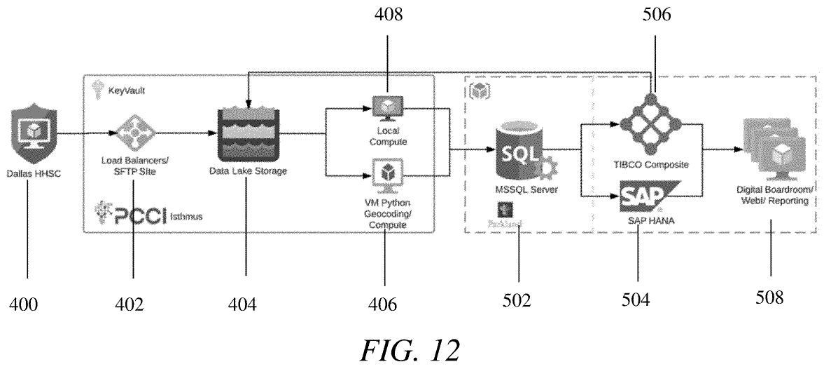

FIG. 12 is a simplified block diagram of the hardware components of an embodiment of the personal pandemic proximity index system and method according to the teachings of the present disclosure. Raw data is pulled from Dallas County Health and Human Service Commission, as known as Dallas HHSC 400. The data is then transferred via Secure File Transfer Protocol (SFTP) site or load balancers 402 into the PCCI Isthmus system. The data is stored in a data lake storage module 404 and controlled, processed, or managed via local compute modules 408 or a VM python geocoding/compute module 406. The data in the PCCI Isthmus system is then transferred to a database management system include Microsoft SQL Server 502, SAP HANA 504, TIBCO Composite 506, and others. The data is analyzed or summarized using a data analytics system (e.g., digital boardroom/web/reporting 508).

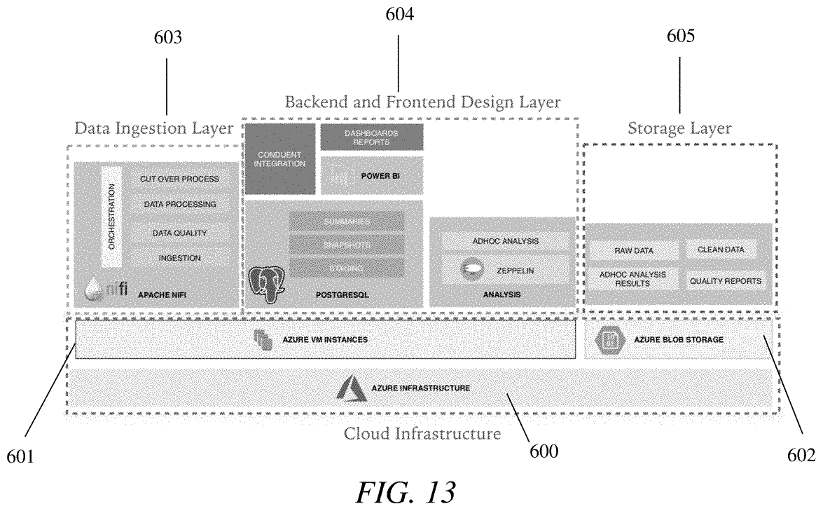

FIG. 13 is a simplified block diagram of the exemplary operating environment of the personal pandemic proximity index system and method. The cloud infrastructure 600 includes three main parts: data ingestion layer 603, back-end and front-end design layer 604, and storage layer 605, and the cloud infrastructure is hosted, for example, on the Microsoft Azure Cloud. The data ingestion layer and the back-end and front-end design layer are in a Azure Virtual Machine Instances 601. Every instance of Azure Virtual Machine is a virtual machine whose specifications are determined by the instance size a user selected when Azure Virtual Machines are used. By hosting everything on a single platform, the dashboard is a streamlined process for ingesting, cleaning, analyzing, and presenting the data.

Through an automated data ingestion engine using, for example, Apache NiFi, raw data is received from a variety of data sources using, for example, File Transfer Protocol (FTP), Simple Object Database Access (SODA)/Application Program Interface (API), client URL (cURL), and other methods.

Apache NiFi is an easy-to-use, powerful, reliable data stream tool for data processing and distribution. It can interface with external various data sources, such as MySQL, Oracle, and the like, and can provide a visual web user interface. Based on the Apache NiFi technology, a visual WEB graphical interface can be provided for a user, so that programming based on a flow can be completed through dragging, connecting and configuring the data. The data flow tool NiFi can automatically load relational database data into objections and relations of a graph database by calling a graph database model interface. For example, when an icon is triggered externally, the data processing module in the data streaming platform may detect the triggering operation, so as to execute the generating operation of the NiFi data flow so that the data can be imported into the graph data module.

Data is automatically pulled from the data sources on a regular or periodic basis to ensure that the current data is the up to date. Real-time data, if available, may also be received and used for analysis. The data sources may include (for data related to, for example, Dallas County, Tex.) Texas Education Agency, the Centers for Disease Control and Prevention, the Census, Feeding America, Department of State Health Services, Dallas Independent School District, Dallas Police Department, Texas Department of Family and Protective Services, Neighborhood Atlas, County Health Rankings & Roadmaps, Centers for Medicare and Medicaid Services, Housing and Transportation Affordability Index, Texas Health and Human Services, U.S. Department of Housing and Urban Development, Dallas County Votes, etc.

The back-end and front-end design layer may use a data presentation/interface tool, for example, the Power BI dashboard tool, to pull data from the database management system via a gateway. Power BI is a Microsoft product that can be easily integrated with the Azure platform. The back-end and front-end design layer conducts ad hoc analysis via data analysis application, such as Zeppelin. With ad hoc analysis, users can extract the insight they need to analyze the disease related questions without having to involve the IT department. An ad hoc report makes it easy for a non-technical audience to understand and utilize a disease report. The storage layer is based on Azure Blob storage 602, which stores raw data, clean data, ad hoc analysis results, and quality reports.

Although this disclosure specifically references the Covid-19 disease and its variants, the system and method described herein are applicable to the analysis of other infectious diseases.

The features of the present invention which are believed to be novel are set forth below with particularity in the appended claims. However, modifications, variations, and changes to the exemplary embodiments described above will be apparent to those skilled in the art, and the system and method described herein thus encompasses such modifications, variations, and changes and are not limited to the specific embodiments described herein.

What is claimed is:

1. A system comprising:
a data ingestion logic module configured to receive pandemic data associated with susceptible, exposed, infected, recovered, and vaccinated people in a community;
a data processing module configured to clean and pre-process the received pandemic data;
a data analysis logic module configured to weigh, normalize, calculate, and use artificial intelligence to analyze the processed pandemic data;
wherein the data analysis logic module includes a herd immunity module configured to determine values associated with an accurate herd immunity estimation; and
a dashboard configured to visually present values for a plurality of indicators associated with herd immunity estimation.

2. The system of claim 1, wherein the herd immunity module is configured to estimate a value for herd immunity based at least in part on an equation HI=1−S/T=1−(T−(θ[I+R]+[I−λ]))/T
where,
HI is the level of herd immunity,
S is the number of people susceptible to a disease,
T is the total number of people in an area,
V is the number of vaccinated cases,
I is the number of measured infected cases within a time window,
R is the number of measured recovered cases within a time window,
θ is Adjusted Incidence Rate Ratio, wherein θ=the estimate of total infected cases/the number of measured infected cases, and
λ is an overlap percent representing the percentage of people who are vaccinated who had the disease previously.

3. The system of claim 2, wherein the herd immunity module is configured to statistically match total confirmed cases and vaccinated cases on the basis of demographic information and geocoded to be in a designated area to account for an overlap between infected cases and vaccinated cases.

4. The system of claim 1, wherein the herd immunity module is configured to account for waning factors, including the time elapsed since the last vaccine dose, the number of vaccine doses, or the time after a prior infection.

5. The system of claim 1, wherein the data ingestion logic is configured to receive real-time and non-real time data from a variety of selected sources, and the data processing module includes a data extraction module, a data cleaning module, and a data manipulation module.

6. The system of claim 1, wherein the data ingestion logic is configured to receive pandemic data including vaccination data, testing data, clinical data, claims data, demographic data, comorbidity data, area deprivation index, mobility data, and recent incidence rata data, from both publicly available and proprietary or licensed data.

7. The system of claim 1, wherein the data analysis logic module is configured to incorporate at least one of machine learning, artificial intelligence, and natural language processing techniques to analyze the pandemic data.

8. The system of claim 1, wherein the dashboard is configured to generates at least one of a heat map, ranked list, scatter plot, and another designated visualization to display graphical data related to the accurate herd immunity estimation values.

9. The system of claim 1, wherein the dashboard is configured to generate a map with overlay layers that show locations of hospitals, testing centers, and vaccinations to assess community needs.

10. The system of claim 1, wherein the dashboard is configured to use a Power BI dashboard tool and set up four dashboards including education, community needs, health, and economy and public safety.

11. A method comprising:
receiving pandemic data associated with susceptible, exposed, infected, recovered, and vaccinated people in a community;
weighing, normalizing, calculating, or using artificial intelligence to analyze the received data and determining values associated with an accurate estimation of herd immunity; and
presenting values for a plurality of indicators and data related to the accurate estimation of herd immunity.

12. The method of claim 11, wherein the received data is calculated based on an equation HI=1−S/T=1−(T−(θ[I+R]+[I-λ]V))/T
where,
HI is the level of herd immunity,
S is the number of people susceptible to a disease,
T is the total number of people in an area,
V is the number of vaccinated cases,
I is the number of measured infected cases within a time window,
R is the number of measured recovered cases within a time window,
θ is Adjusted Incidence Rate Ratio, wherein θ=the estimate of total infected cases/the number of measured infected cases, and
λ is an overlap percent representing the percentage of people who are vaccinated who had the virus previously.

13. The method of claim 11 further comprising statistically matching the data for total confirmed cases and vaccinated cases on the basis of demographic information and geocoding to be a designated area to account for an overlap between infected cases and vaccinated cases.

14. The method of claim 11, further comprising accounting for waning factors, including the time elapsed since the last vaccine dose, the number of vaccine doses, or the time after a prior infection.

15. The method of claim 11, further comprising receiving real-time and non-real time data from a variety of selected sources, and extracting, cleaning, and manipulating the received data according to a predefined script.

16. The method of claim 11, further comprising processing data using at least one of machine learning, artificial intelligence, or natural language processing techniques.

17. The method of claim 11, wherein receiving pandemic data includes receiving vaccination data, testing data, clinical data, claims data, demographic data, comorbidity data, area deprivation index, mobility data, and recent incidence rata data, from both publicly available and proprietary or licensed data.

18. The method of claim 11, wherein presenting values comprises generating a map with overlay layers that show locations of hospitals, testing centers, and vaccinations to assess community needs.

19. The method of claim 11, further comprising a Power BI dashboard setting up four dashboards including education, community needs, health, and economy and public safety.

20. The method of claim 11, wherein presenting values comprises generating and displaying at least one of a heat map, ranked list, scatter plot, and another designated visualization to display graphical data related to the accurate herd immunity estimation values.
